(19) United States
(12) Patent Application Publication
(10) Pub. No.: US 2013/0328234 A1
(43) Pub. Date: Dec. 12, 2013

(54) POLYTITANIC ACID ESTERS AND USE THEREOF TO PRODUCE IMPLANTABLE, OPTIONALLY ABSORBABLE FIBERS

(71) Applicants: Rainer Jahn, Zell (DE); Miranda Rothenburger-Glaubitt, Margetshochheim (DE); Walther Glaubitt, Margetshochheim (DE); Joern Probst, Kuernach (DE)

(72) Inventors: Rainer Jahn, Zell (DE); Miranda Rothenburger-Glaubitt, Margetshochheim (DE); Walther Glaubitt, Margetshochheim (DE); Joern Probst, Kuernach (DE)

(73) Assignee: Fraunhofer-Gesellschaft zur Förderung der angewandten Forschung e. V., Muenchen (DE)

(21) Appl. No.: 13/985,755

(22) PCT Filed: Feb. 14, 2012
(86) PCT No.: PCT/EP2012/052513

§ 371 Date: Aug. 15, 2013

(30) Foreign Application Priority Data
Feb. 17, 2011 (DE) 10 2011 011 544.7

(51) Int. Cl. A61L 31/14; D01F 11/00

(52) U.S. Cl. CPC A61L 31/148; D01F 11/00
USPC 264/205; 556/55; 106/287.19

(57) ABSTRACT

The invention relates to polytitanic acid esters having linear cross-linking structures, obtainable by reacting a titanium compound that is soluble in an alcohol, in water or in a mixture of water and alcohol and that has the composition (I)

Ti(R)ₘXₙ (I),

wherein the groups R independently mean alkoxy, carboxy, or OH, X means halogen, m=0 to 4, n=0 to 4, and m+n=4,
with a compound (II) that contains the following structural unit:

[O═CR⁴—(CR⁵R³)ₚ—CR¹R²—O]⁻,

wherein p is 0 or 1 and wherein

(a) R¹ and R² are independently selected from among hydrogen, alkyl and alkenyl, R³ and R⁵ are independently selected from among hydrogen or an unsubstituted or substituted alkyl or alkenyl, R⁴ is OH or O(M^{x+})_{1/x}, wherein M is a metal or ammonium and x is 1 or 2, or

(b) R¹ and R² together mean ═O and R³, R⁵ and R⁴ are independently selected from among hydrogen, alkyl and alkenyl,

wherein the titanium compound (I) is reacted with compound (II) at a molar ratio of 1 to 0.5-1.9, with respect to an unbridged titanium compound, i.e., a (monomeric) titanium compound containing a single titanium atom, with the stipulation that, if compound (I) has no more than 2 hydroxy groups, and if the reaction occurs in a water-free medium, the product formed by the reaction is then brought in contact with water in such a way that a hydrolytic condensation reaction of the produced titanium compound(s) occurs in all cases.

Such polytitanic acid esters can be spun in the form of viscous masses to create fibers.

The present invention relates to polytitanic acid esters as well as a spinnable mass that can be produced by means of hydrolysis and condensation of a titanium coordination compound having fewer than two 2-hydroxy carboxylic acid anions per titanium atom and which contains said mass. In particular, the mass can contain water-soluble poly propionic acid-2-oxo-titanium oxide hydrate or a salt thereof. Fibers spinnable thereof can be used as resorbable implant material in the field of medical engineering, for example as scaffolds for the regeneration of bone, cartilage or soft tissue cells.

Resorbable implants have been used in the field of implantology for many years. Resorbable biomaterials became known in connection with the development of resorbable sutures on the basis of synthetically manufactured polylactides (PLAS) and polyglycolides (PGLAs) in the 1970s of the last century. In a next generation, this material was used to produce implants such as screws and plates to fixate tissue and bones. As the know-how about the material properties, degradation behavior and feasibility of different clinical indications increased, the first vessel supports (stents), soft tissue reinforcement implants (e.g. hernia repair mesh patches) and membrane systems, such as they are used among other things for jaw surgeries, were successfully manufactured in the 1990s. Other, more recent examples include hydrogen-filled tubes used as so-called nerve conduction guides for neuronal regeneration or cell carrier structures used in the field of tissue engineering.

Carrier materials used for tissue engineering usually contain organic, resorbable polymers (PLAs/PGLAs) as basic component. The original stability of the structure and hence the shape is initially achieved with the resorbable supporting body. After a certain proliferation phase of bone, cartilage or soft tissue cells, their own load bearing capacity increases and the carrier synthetic of the supporting body in turn is almost dissolved to the point where only pure cell tissue or bone material is present. The problem with resorbable implants according to the prior art is the fact that the implant is identified as foreign body by the recipient due to its material, thus inducing a sterile immune response. Indeed, this does generally not cause rejection reactions like those observed with allogeneic (i.e. originating from a human donor) or xenogeneic (originating from an animal donor) organ implants; however, it can interfere with the adhesion and growth of the body's own cells in the region of the implant.

Resorbable implants such as suture materials made of PLA have been improved with the application of a very thin, non-resorbable titanium coat. The non-resorbable coat only partly covers the resorbable material. The adhesion and growth of the body's own cells are promoted in the coated area, thus facilitating a faster healing process. The resorption is slowed down on the coated part of the implant, see EP 1 752 167 E1.

Biodegradable or resorbable fibers with the formal chemical composition Siₙ(OH)₂ₓO₂ₙ₋ₓ were produced at the Fraunhofer Institute for Silicate Research. The mechanical properties of composite materials produced with the fibers, for example a resorbable wound dressing, are clearly superior to those of pure polymer compact materials. For instance, the wound dressing maintains its support function for a considerably longer period of time than a traditional organic implant. The cell adhesion and proliferation are not impaired. The degradation rate (solubility) is determined by the silanol content which can be adjusted with the manufacturing parameters (water content, storage).

Fibers made of commercially available titanium-bis-(ammonium lactato)-dihydroxide in combination with polyvinyl alcohol (PVA) as spinning additive can be produced by means of an electrospinning procedure. TiO₂ fibers suitable for photocatalytic purposes (K. K. Nakane et al., Journal of Applied Polymer Science, Vol, 104, 1232-1235 (2007)) were produced from said fibers by means of calcination. The electrospinning procedure was used by T. Schiestel at the Fraunhofer institute for Interfacial Engineering and Biotechnology (IGB) for the manufacture of ultra-thin fibers. In the process, the jet of a fluid is accelerated in the electrical field and continuously split into additional rays which mutually repel each other due to the like charges. If polymer solutions are used for this purpose, the solvent evaporates during the spinning process, ultimately creating polymer fibers with diameters in the sub-micrometer and nanometer range. Electrospinning can be used for a variety of materials including polyvinylpyrrolidone (PVP), PEO or PLA. By adding inorganic precursors to the spinning solution and subsequent sintering, it is also possible to produce purely inorganic fibers.

Other methods for the manufacture of TiO₂ fibers have been disclosed. Based on the teachings of the patents U.S. Pat. No. 6,191,067 and U.S. Pat. No. 6,409,061, alcoholic solutions of titanium alkoxides (with and without complexation by β-diketones of the formula R⁴COCH₂COR⁵, wherein R⁴ and R⁵ represent alkyl or alkoxy groups, or alkyl salicylates) are hydrolyzed and reduced. The resulting polymer is taken up with tetrahydrofuran and spun. The fibers are treated with water vapor and calcined. A similar disclosure was made in U.S. Pat. No. 4,166,147. JP 62223323 (1987) describes an alcoholic titanium oxide solution which is hydrolyzed and condensed with hydrochloric acid. Said solution can allegedly be used to spin fibers. They are heated to obtain titanium oxide fibers. A similar method is described in EP 1 138 634 B1. However, the information contained in this printed document was not reliably reproducible. In fact, the inventors of the present application determined that the aqueous hydrolysis of isopropoxy titanate under the conditions described in EP 1 138 634 B1 yields a suspension which cannot be reduced to a spinnable mass. This is not surprising because it is generally known that titanium alkoxides instantly condense hydrolytically with the addition of water, wherein ring-shaped or three-dimensional clusters are formed.

Textile structures made of resorbable fibers are frequently used as medical implants to help support or replace soft and hard tissue. The suitable process management and selection of the fiber material makes it possible to achieve a large variety of surfaces, porosities and mechanical anisotropies to mimic the unique structural and mechanical properties of biological tissues. Current applications include wound care (fleece), wound closure (suture materials), hernia repair mesh patches and receptacles (knits) as well as cell carrier systems to grow bone, skin, cartilage, tendons or liver tissue in vitro.

Based on its high corrosion-resistance and excellent biotolerance, titanium is a widely used material for resorbable implants. For hips alone, more than one million endoprostheses with titanium shafts and acetabular cups made of titanium are implanted annually around the world. Titanium owes its favorable biotolerance to the only several nanometers thin oxide hydrate layer at the surface. Human osteoblast cells literally spread out on it, firmly anchoring with the material, thus creating direct contact with the bone. Fibroblasts propagate unhindered and grow all the way to edges of implants made of titanium passivated with titanium oxide, see Kohler S. T., Retemeyer K., Berger G., Studies investigating the adhesion promotion of bio-vitroceramics and titanium in bones of animals, Journal of Experimental Surgery 14 (1981) 139-143. Studies investigating the brain tissue of rabbits following titanium implants did not reveal any degenerative alterations, see Hirai H., Okumura A., Goto M., Katsuki T., Histologic study of the bone adjacent to titanium bone screws used for mandibular fracture treatment, Journal of Oral and Maxiliofacial Surgery 59 (2001) 531-537 and Morra M., Cassinelli C., Cascarde G., Cahalan P., Cahalan L., Fini M., Giardino R., Surface engineering of titanium by collagen immobilization. Surface characterization and in vitro and in vivo studies, Biomaterials 24 (2003) 4639-4854.

Derivatives of lactic acid (2-hydroxypropionic acid) are likewise widely used in the field of medical engineering. in this respect, reference is made for example to Lucke, A., J. Tessmar, at al. Biodegradable poly(D,L-lactic acid)poly(ethylene glycol)-monomethyl ether diblock copolymers: structures and surface properties relevant to their use as biomaterials, Biomaterials 21(23) (2000) 2361-70. PLA is used for a number of medical applications due to its biocompatibility and degradability in the human body. As an example, PLA, often combined with a copolymer, is a suitable suture material. However, it is also used to manufacture nails and screws, plates or slants. Irrespective of the chemical composition and porosity, FLA can remain in the body for a few months to several years, until it is broken down.

Titanium complexes with lactic acid have already been described in U.S. Pat. No. 2,870,181; their usability as gelling agent for polyhydroxy compounds such as starch and polyvinyl alcohol is discussed there.

The invention was based on the task to develop a resorbable implant material which on the one hand can be brought into the form of fibers and on the other hand combines several benefits of resorbable organic implant materials made of poly-(α-hydroxy carboxylic acids) such as PLA on the one hand and titanium oxide hydrate surfaces on the other hand.

Many attempts at finding such a material failed. The inventors complexed a titanium starting material such as titanium tetraethylate with an α-hydroxy carboxylic acid to create a hydrolytically stable oxo compound of titanium which can hence be used in aqueous media. Said compound was mixed with a known spinning additive such as polyvinyl alcohol, polyacrylic acid or polyethylene oxide, and an attempt was made to form fibers hereof by extruding the created mass through nozzles. As well, commercially available titanium-bis(ammonium lactato)-dihydroxide was processed with spinning additives and tested. Yet all the spinning tests, irrespective of the used spinning additive, were not satisfactory because of frequent thread tear-off. Moreover, the addition of said types of spinning additives is potentially risky, but at least not optimal, because additional additives are added to the future fiber whose biotolerance is not always clarified. The retroactive removal of the potentially problematic additive by way of extraction and with the conservation of the form of the fibers was not successful. It was only possible to remove spinning additives by means of adding heat, which however created insoluble, non-resorbable TiO₂.

Therefore, the inventors attempted to produce fibers without spinning additives.

Two titanium compounds are known in which the titanium atom is complexed with the α-hydroxy carboxylic acid lactic acid, namely titanium-bis-(ammonium lactato)-dihydroxide (see H. Mockel at al., J. Mater, Chem., 1999 (99), 3051-3056), marketed as 50% aqueous solution, and ammonium trilactatotitanate (Inorg. Chem. 2004, vol. 43, 4546-4548). Based on information from the authors, the first of the two molecules has a monomeric structure with two chelating complexing ammonium lactate anions and two bound hydroxyl groups. Based on X-ray structure analysis, the latter molecule, which is also water-soluble, exists at least in the solid state in the form of a monomeric compound complexing with three lactate molecules, wherein all three lactate molecules form a 5-membered ring with the titanium atom via bidentate oxygen. Accordingly, it does not comprise any potential substituents, e.g. an OH or alcoholate group which would allow oligomerization. Therefore, said compound had to be discounted from the start. In contrast, titanium-bis-(ammonium lactato)-dihydroxide carries two hydroxy groups and can therefore condensate to form polymer chains. They are necessary to obtain a spinnable material without spinning additives. Consequently, as expected, a highly viscous mass was created by reducing an aqueous solution of titanium-bis-(ammonium lactato)-dihydroxide, from which threads were drawn by means of a glass wand. However, the thin threads instantly contracted and formed a short, thick strand. Therefore, this type of mass is not suitable for the manufacture of fibers made with an aqueous/alcoholic solution through spinning by means of spinning nozzles. It is likely that no or only inadequate linear polymer structures are formed during the evaporation and augmentation of the concentration of the solution. The OH groups at the Ti atom apparently only condensate to a minor degree due to two ammonium lactato substituents at the central Ti atom, and the steric hindrance is so strong that the oligormerization is insufficient to achieve a stable thread formation.

Surprisingly, the inventors still managed to solve the task of the invention and to obtain a spinnable mass with the desired properties, namely with the reaction of a reactive titanium compound of the formerly quadrivalent titanium that is soluble in alcohols or water with a compound (II) comprising the following structural unit:

[O═CR⁴—(CR⁵R³)_{P}—CR¹R²—O]⁻,

provided the titanium compound is reacted with the compound (II) at a molar ratio of 1 to 0.5-1.9, preferably of 1 to 0.7-1.5, with respect to an unbridged titanium compound, i.e., a (monomeric) titanium compound containing a single titanium atom, and optionally a subsequent hydrolysis of the formed chelate complex to facilitate the required condensation processes. If the starting material does not yet comprise any Ti—OH groups, water will be added for this purpose during or after the reaction with the compound containing the mentioned structural unit. Titanic acid esters with at least in part only one of the structural units defined above per titanium atom as well as hydroxyl groups bound at the titanium atom are formed in the process. Titanic acid esters with only one structural unit as described above have the theoretical formula Ti(O═CR⁴—(CR⁵R³)ₚ—CR¹R²—O)(OH)₃. They can form oxo bridges with other titanium atoms in the known fashion, meaning that they are free for at least partial linear polymerization. The polymeric titanic acid esters are created with this condensation reaction; they are available as viscous mass from which stable threads can be spun by means of a spinning nozzle, optionally after removing part of the solvent in which they were produced.

In a first variant (variant “a”), the structural element is a component of a hydroxy carboxylic acid. In these cases, the substituents and indices are defined as follows: R¹ is hydrogen, alkyl or alkenyl, preferably hydrogen, C₁-C₆-alkyl or C₁-C₆-alkenyl, particularly preferably hydrogen or C₁-C₄-alkyl, R² is hydrogen, alkyl or alkenyl, preferably hydrogen, methyl or ethyl, and R³ and R⁵ independently are hydrogen or an unsubstituted alkyl or alkenyl or an alkyl or alkenyl preferably substituted with OH, more preferably having 1 to 4 carbon atoms each. R⁴ is a hydroxy group or a corresponding salt OM, wherein M=monovalent cation such as Li, Na, K, NH₄, or OM_{1/2}, wherein M bivalent cation such as Mg or Ca, wherein such a cation can then coordinate two molecules of the structural element. The index p can be 0 or 1, where it is beneficial in case p=1, that at least one of the groups R³ and R⁵ is not hydrogen. Preferably, R⁵ is a methyl or ethyl_{;} optionally substituted with hydroxy in this case.

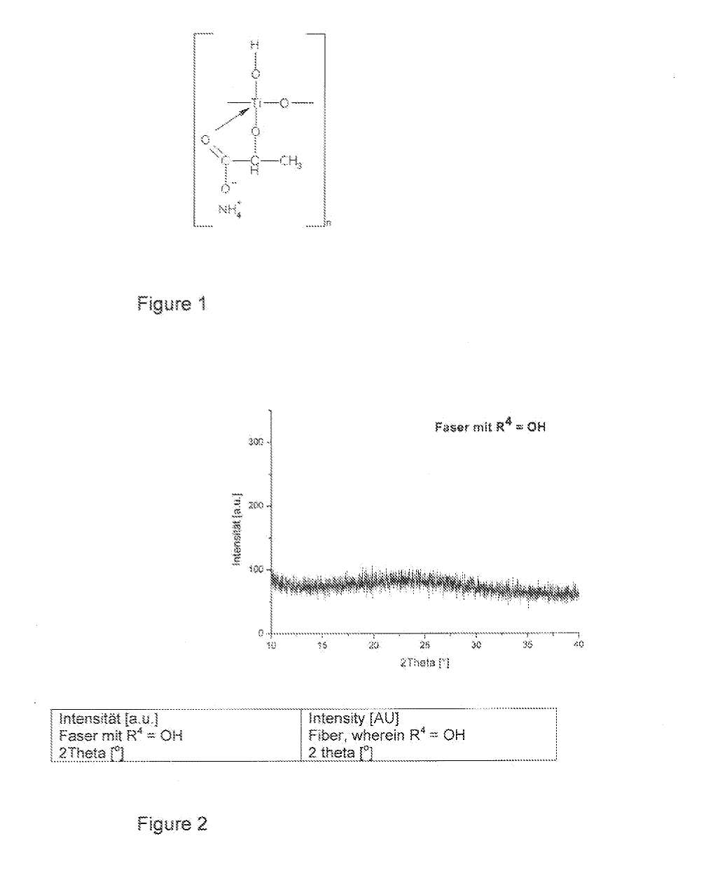

If an acid is reacted with the titanium starting compound according to this first variant, it likely forms a five- or six-member chelate ring with the titanium atom, in that it is complexed at the titanium atom via the anion of the hydroxy group and the C═O group of the carboxy group, see FIG. 1. This assumption is based on a number of reasons: firstly, this type of complexation is mentioned in the literature for the two lactic acid groups of the molecule bis(ammonium lactato)-titanium dihydroxide (see Möckel et al., loc. cit.), and secondly, the inventors have measured an extremely low pK_{S} for compounds of this type. The proton of the carboxy group is thought to be responsible for this phenomenon, which can optionally be successfully exchanged with a different cation such as sodium, potassium or ammonium by reaction with a base.

In preferred embodiments of the first variant of the invention, the compound with the mentioned structural unit is glycolic acid (2-hydroxyethanoic acid), lactic acid (2-hydroxypropionic acid), 2-hydroxybutyric acid, 2-hydroxyisobutyric acid or 2,2-bis(hydroxymethl)propionic acid. The reaction of one of these acids with titanium ethylate or similar, optionally after hydrolysis of the remaining groups present at the titanium atom, results in a product which can likely be referred to as carboxylic acid-2- or 3-oxotitanate-trihydroxide, for example as ethanoic acid-, propionic acid or 2-hydroxymethyl-2-methyl-propionic acid-3-oxotitanate-trihydroxide. The compound created during the exchange of the free acid proton with an alkali or ammonium cation can then be referred to as alkali or ammonium carboxylate-2-oxotitanate trihydroxide. A spinnable mass can be obtained both from compounds with a complexed carboxylic acid group as well as from compounds with a complexed cation carboxylate group by way of hydrolysis, said mass likely containing the corresponding poly(carboxylic acid-2-oxo-titanium oxide hydrate) and poly(alkali- or ammoniumcarboxylate-2-oxotitaniurn oxide hydrate) created with linear polymer chains. Said types of polymers are herein referred to as polytitanic acid esters.

In a second variant (“variant b”), the structural unit is part of a keto- or aldo-carboxylic acid. In this case, R¹ and R² together mean ═O, R⁴ is alkyl or alkylen, preferably having 1 to 16 carbon atoms and hydrogen, while the remaining groups and indices have the same meaning as in the first variant. In this variant, R³ and R⁵ can advantageously both be hydrogen. Examples of acids that can be used include pyruvic acid (2-oxopropanoic acid) and glyoxylic acid (oxoethanoic acid).

In all embodiments, the alkyl, alkenyl and alkylen groups of the present invention can be provided independently from each other as straight chains, in branched or cyclical form. They can be substituted or unsubstituted.

Irrespective of the above, the index p is 0 in a preferred embodiment relating to both variants.

All embodiments mentioned above can be combined with each other, unless they mutually exclude each other.

Any titanium compounds in which titanium is present in formally quadrivalent form are suitable as titanium-based starting compound. Compounds in which no more than 2 OH groups are present are preferred. Purchasable titanium alicylates (titanium alkoxides) such as titanium ethylate Ti(OC₂H₅)₄ are advantageous. Titanium compounds without free hydroxy groups are particularly preferred.

Aside from the combination with the mentioned structural unit, other monocarboxylic acids such as ethanoic acid or propionic acid can be added to the reaction.

The molar ratio of a (monomeric) titanium compound to the compound (II) containing the mentioned structural unit is preferably in the range of 1 to 0.7-1.5, more preferably in the range of 1 to 0.9-1:1 and most preferably in the range of 1:1 in the process, it was determined that in cases where less than 0.5 mol, in some cases also less than 0.7 mol of the compound containing the structural unit was used per mol of titanium, no reaction product soluble in water was obtained, and it was therefore impossible to draw threads. With a ratio of more than 1.9 mol of compound containing the structural unit per mol of titanium, no masses were produced from which it was still possible to draw threads. in the range between 1.5 and (frequently) 1.8, at least up to about 1.7 mol of the compound containing the structural unit per mol of titanium, it was indeed possible to draw threads, although they were not stable in all cases; some of them were impossible to dry and/or contracted and formed drops. Therefore, it is preferred according to the invention, to select the ratio of compound containing the structural unit such that it does not exceed 1.6, preferably does not exceed 1.5 mol per mol of (monomeric) titanium compound.

If a monocarboxylic acid is additionally added to the reaction, it is recommended that the molar ratio of (monomeric) titanium compound to the sum of compound (II) and the monocarboxylic acid is likewise in the upper range for the ratio of titanium compound to compound (II) or that it only falls slightly short of it.

Small angle X-ray analyses of preparations containing an equimolar ratio of lactic acid and titanium ethylate reveal an oligomeric structure with a size of 3 nm. Indeed, these kinds of colloids do not crystallize when reduced; however, a person skilled at the art had to fear that they would condensate to become a three-dimensional unspinnable gel during the loss of solvent. This was expected especially because the suspended substance comprises three condensation centers (hydroxy groups) per central atom, (this is generally the case for compounds that only carry one chelate substituent at the titanium atom). It was therefore surprising that solutions of titanium compounds that meet the criteria outlined above can be reduced to stable, spinnable masses. The inventors assume that a poly(carboxylic acid-2-oxo-titanium oxide hydrate) with linear polymer chains is formed in the respective hydrous or aqueous solution, said polymer chains neither crystallizing nor cross-linking.

The reaction generally takes place in suspension, preferably in an alcohol or an alcohol-water mixture and optionally directly in water. Alkyl alcohols that are liquid at room temperature and optionally at temperatures up to at least 60° C. such as methanol, ethanol, n- or i-propanol or n-, i- or t-butanol are preferably used as alcohols. However, optionally (and less preferably) other polar solvents alone or mixed with the ones mentioned above, can be used. Examples are ketones such as acetone. Irrespective of the selection of the solvent, it can additionally contain acids, in particular organic acids such as lactic acid or a different acid usable for the invention, or a base such as e.g. ammonia.

At least in the event that the titanium-based starting compound does not comprise any free hydroxy groups, the presence or subsequent addition of water is compulsory for the hydrolysis. This is also recommended if the titanium-based starting compound does not comprise more than two free hydroxy groups. In order to prevent the formation of titanium dioxide (anatase), the hydrolysis is preferably carried out at a temperature ranging between 10 and 35° C., in particular at room temperature. The product is present in the selected solvent (mixture) in the form of a sometimes turbid, sometimes clear, gel-like solution. Said solution can be adjusted to the desired solid matter concentration and viscosity as needed, e.g. by means of augmenting the concentration (where the well-established removal with a rotary evaporator at reduced pressure can be used) and optionally by means of dilution. It is advantageous to evaporate the solvent until a viscous mass with a solid matter content of approximately 20-50 percent in weight, preferably 30-35 percent in weight is created, wherein the solid matter content here is defined as the (theoretical) titanium dioxide content. Excessively high temperatures should again be avoided for this step; it is advantageous to work with bath temperatures in the range 30-70° C., more preferably of about 5° C.

For the manufacture of the fibers, the viscous mass can be extruded through nozzles or drawn to create threads with a different method. For this purpose, mainly nozzles with a diameter between 50 μm and 1000 μm are considered. The viscosity ranges between 30 and 250 Pa·s (20 ° C.). Preparations with higher viscosities can also be spun through smaller nozzle diameters (150 μm) after they have been heated to 40° C. By adjusting the spinning temperature, the viscosity (the range of 30-50 Pa·s has proven to be advantageous), the concentration of the spinning mass and the nozzle diameter, the diameters of the obtained fibers can be adjusted to a wide range, wherein a range from 5 to 200 μm is particularly advantageous. The created fibers can be wound up as continuous fibers, optionally after having been dried or, be stored otherwise, e.g. after having been cut, for instance in the form of a fleece. A drop distance advantageously with a length of several decimeters to meters is suitable to achieve the initial dryness.

The fibers obtained by means of spinning are readily soluble in water and can dissociate into colloidal titanium oxide hydrate, the same compound as the one present on the surface of titanium implants, as well as the chelating compound or its salt if its acidic proton was exchanged for a salt cation. If the chelating compound is a biocompatible or even a resorbable compound such as lactic acid or a salt thereof such as ammonium lactate, it can dissociate into catabolic products such as sodium or calcium lactate, optionally even by interacting with the body's own substances. These are substances generally recognized as safe by the Food and Drug Administration (FDA). These kinds of safe catabolic products can optionally also be created with the addition of calcium or magnesium hydroxide to the spinning preparations.

The water solubility of the obtained fibers can be modified by exposing them to a specific temperature regimen. If they are exposed to more heat, their water solubility decreases. Heating them for several hours at temperatures ranging from 100 to 300° C., e.g. at 200° C. for approximately 20-30 hours, can render them completely insoluble in water.

Numerous in vivo and in vitro experiments have already confirmed the biocompatibility of amorphic titanium oxide hydrate on titanium implants. While fibers made of pure organic polymers such as e.g. polyglycosides, D/L-polylactides or inorganic calcium phosphate recrystallize and may hence induce foreign body rejection reactions and/or are subject to degradation, where the degradation products cause an intolerable pH shift toward acidic values at the implant site, the fiber according to the invention can be produced in a pH-neutral form in one embodiment of the invention, such that it is neither expected to have any negative impacts on the tissue milieu with respect to its degradation products nor to induce any foreign body reactions. If the acidic material is used for the fiber instead, the acid group can be utilized for the linkup of active substances.

Below, the invention is explained in more detail by means of examples.

1. Manufacture of Spinnable Masses

### Example 1.1

Variant a; R⁴=OH; R¹=H: R²=CH₃; p equals 0; lactic acid content: 1 mol; high water content.

230.35 g (5.00 mol) of ethanol are added to 233.0 g (1.00 mol) of titanium ethylate at room temperature. After a clear solution has formed, 105.98 g (1.00 mol) of a lactic acid solution 85% are added at room temperature. The reaction mixture reacts slightly exothermic. After cooling down to room temperature, 333.33 g (18.5 mol) of water are added at once, resulting in turbidity of the gel and formation of white precipitate. The reaction mixture is stirred for 24 h at room temperature; the precipitate dissolves again in the process. The solvent is removed from the mixture with a rotary evaporator in the water bath (50° C.) at a pressure of up to 40 mbar until a tough mass is created.

### Example 1.2

Example 1.1 was repeated with the stipulation that the corresponding molar ratio of glycolic acid was used instead of lactic add.

### Example 1.3

Variant a; R⁴=OH; R¹=H: R²=CH; p equals 0; lactic acid content: 1 mol; low water content.

230.35 g (5.00 mol) of ethanol are added to 233.0 g (1.00 mol) of titanium ethylate at room temperature. After a clear solution has formed, 105.98 g (1.00 mol) of a lactic acid solution 85% are added at room temperature. The reaction mixture reacts slightly exothermic. After cooling down to room temperature, 9.01 g (0.5 mol) of water are added at once, resulting in turbidity of the gel and formation of white precipitate. The reaction mixture is stirred for 24 h at room temperature; the precipitate dissolves again in the process. The solvent is removed from the mixture with a rotary evaporator in the water bath (50° C.) at a pressure of up to 40 mbar until a tough mass is created.

### Example 1.4

Variant a; R⁴=OH; R¹=H; R²=CH₃; p equals 0; lactic acid content: 0.75 mol; low water content.

230.35 g (5.00 mol) of ethanol are added to 233.0 g (1.00 mol) of titanium ethylate at room temperature. After a clear solution has formed, 79.48 g (0.75 mol) of a lactic acid solution 85% are added at room temperature. The reaction mixture reacts slightly exothermic. After cooling down to room temperature, 9.01 g (0.5 mol) of water are added at once, resulting in turbidity of the gel and formation of white precipitate. The reaction mixture is stirred for 24 h at room temperature; the precipitate dissolves again in the process. The solvent is removed from the mixture with a rotary evaporator in the water bath (50° C.) at a pressure of up to 40 mbar until a tough mass is created.

### Example 1.5

Variant a: R⁴=OH; R¹=H; R²=CH₃; p equals 0; lactic acid content: 0.5 mol; low water content.

230.35 g (5.00 mol) of ethanol are added to 233.0 g (1.00 mol) of titanium ethylate at room temperature. After a clear solution has formed, 52.99 g (0.50 mol) of a lactic acid solution 85% are added at room temperature. The reaction mixture reacts slightly exothermic. After cooling down to room temperature, 9.01 g (0.5 mol) of water are added at once. resulting in turbidity of the gel and formation of white precipitate. The reaction mixture is stirred for 24 h at room temperature; the precipitate dissolves again in the process. The solvent is removed from the mixture with a rotary evaporator in the water bath (50° C.) at a pressure of up to 40 mbar until a rough mass is created.

### Example 1.6

Variant a; R⁴=OM, wherein M=NH₄⁺; R¹=H; R²=CH₃, p equals 0; lactic acid content: 1 mol; high water content.

230.35 9 (5.00 mol) of ethanol are added to 233.0 (1.00 mol) of titanium ethylate at room temperature. After a clear solution has formed, 105.98 g (1.00 mol) of a lactic acid solution 85% are added at room temperature. The reaction mixture reacts slightly exothermic. After cooling down to room temperature, 333.33 g (18.5 mol) of water are added at once, resulting in turbidity of the gel and formation of white precipitate. The reaction mixture is stirred for 24 h at room temperature; the precipitate dissolves again in the process. The colloidal solution is neutralized with 47.58 g (0.70 mol) of ammonia hydroxide (25%). The reaction mixture heated by the exothermal reaction is stirred until it has cooled down to room temperature. The solvent is then removed from the mixture with a rotary evaporator in the water bath (50° C.) at a pressure of up to 40 mbar until a tough mass is created

The quantity ratios mentioned above were varied in the range of 0.5 to 1.8 mol of lactic acid per mol of titanium compound. It was easily possible to spin threads from masses based on a quantity ratio between 0.7 and 1.5 mol of lactic acid per mol of titanium compound.

### Example 1.7

Variant a; R⁴=OM, wherein M=NH₄⁺; R¹=H; R²=H; p equals 0; lactic acid content: 1 mol; high water content.

Example 1.6 was repeated with the stipulation that glycolic acid was used instead of lactic acid.

### Example 1.8

Variant a: R⁴=OM, wherein M=NH₄⁺; R¹=H; R²=(CH₃; H); p equals 0; lactic acid content: 1 mol: high water content.

The manufacture was analogous to the examples 1.6 and 1.7, wherein however a mixture lactic acid and glycolic acid at a ratio of 1:0.5 was used.

### Example 1.9

Variant (a); R⁴=OM, wherein M=NH₄⁺; R¹=H; R²=CH₃; p equals 0; with the addition of an organic acid (propionic acid),

1 mol (233 g) of titan ethylate are mixed with 230 g of ethanol, 0.75 mol (79.48 g) of lactic acid (85%) are added in approximately 10 portions while stirring. The mixture is then stirred for 2 h. After the addition of 0.25 mol (18.52 g) of propionic acid, the stirring is continued for one more hour. 18.5 mol (333 g) of water are poured in at once while stirring vigorously, whereupon the solution becomes turbid and some gelling is observed. The solvent is removed from the mixture with a rotary evaporator in the water bath (50° C.) at a pressure of up to 40 mbar. It clarifies in the process. 0.19 mol (12.94 g) of ammonia 33% are added and the removal of solvent from the mixture with a rotary evaporator is continued until a tough mass with a viscosity of 40 Pa·s and an oxide content of approximately 36% of titanium dioxide is obtained.

### Example 1.10

Variant a, R⁴=OH; R¹=CH; R²=CH₃; p equals 0; 1.5 mol of 2-hydroxyisobutyric acid; high water content.

7.81 g (0.075 mol) of 2-hydroxyisobutyric acid are added at room temperature to a solution of 11.65 g (0.05 mol) of titanium ethylate in 11.52 g of ethanol. 16.65 g (0.92 mol) of water and 3.41 g (0.05 mol) of ammonia 25% are added to the clear solution. After reduction using the rotary evaporator at 50° C. and 50 mbar, a viscous mass is obtained from which threads can be drawn.

### Example 1.11

Variant a; R⁴=OH; R¹=H; R²=CH₃; R⁵=CH₂OH; p=1; 1.5 mol of 2,2-bis(hydroxymethyl)propionic acid.

10.06 g (0.075 mol) of 2,2-bis(hydroxymethyl)propionic acid are added at room temperature to a solution of 11.65 g (0.05 mol) of titanium ethylate in 11.52 g of ethanol. White precipitate forms which dissolves after the addition of 16.65 g (0.92 mol) of water and 3.41 g (0.05 mol) of ammonia 25%. The solution clarifies completely when heated to 50° C. After reduction using the rotary evaporator at 50° C. and 50 mbar, a viscous mass is obtained from which threads can be drawn.

### Example 1.12

Variant b; R⁴=CH₃, R₁ and R₂ together equal ═O; p equals 0: 1.5 mol of pyruvic acid; high water content.

6.65 g (0.075 mol) of pyruvic acid are added at room temperature to a solution of 11.65 g (0.05 mol) of titanium ethylate in 11.52 g of ethanol. A brown, clear solution is created. After the addition of 16.65 g (0.92 mol) of water and 3.41 g (0.05 mol) of ammonia 25%, a gel is formed which dissolves when heated to 50° C. After reduction using the rotary evaporator at 50° C. and 50 mbar, a viscous mass is obtained from which threads can be drawn at 50° C.

### Example 1.13

Variant b; R⁴equals H; R₁ and R₂ together equal ═O and p equals 0; 1 mol of glyoxylic acid (oxoethanoic acid); high water content,

4.60 g (0.05 mol) of glyoxylic acid are added at room temperature to a solution of 11.65 g (0.05 mol) of titanium ethylate in 11.52 g of ethanol. A yellowish, clear solution is created. White precipitate forms after the addition of 16.65 g (0.92 mol) of water and 3.41 g (0.05 mol) of ammonia 25%. After reduction using the rotary evaporator at 50° C. and 75 mbar, the preparation clarifies and a gel-like, mass is obtained from which short threads can be drawn.

### Reference Example

Hydrolysis of titanium isopropylate without complexing agent.

60.10 g of Ti(OiPr)₄ were added to 14.80 g of 2-propanol and stirred at room temperature. White precipitate is immediately formed after the addition of 7.01 g of water The suspension was kept at room temperature for 1 h and subsequently reduced to dryness. The obtained solid was taken up in approximately 300 mL of THF and the yellowish suspension obtained in this fashion stirred at room temperature for 2 h. The reduction of the suspension using the rotary evaporator resulted in the direct transformation of a suspension to a yellowish solid.

2. Fiber Manufacture

The viscous mass of examples 1.1 to 1.3 was in each case extruded through one or a plurality of 200 μm nozzles at a temperature of 25° C. and a pressure of 20 bar. After dropping 2.5 m, the fibers were reeled onto a rotating cylinder or set down in the form of a fleece using a jig table to horizontal surface movable in 2 dimensions). The fiber diameter generally ranges between 20 and 100 μm.

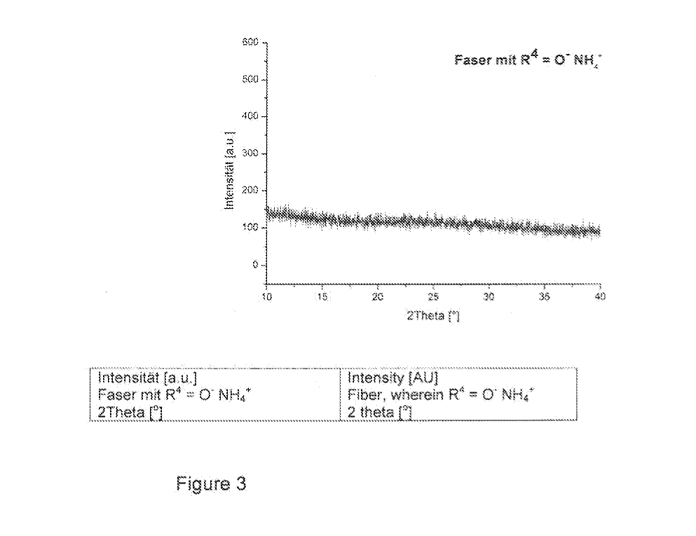

A powder X-ray diffractogram of fibers obtained in this fashion according to example 1.1 is illustrated in FIG. 2 and one according to example 1.7 is illustrated in FIG. 3. Both powder X-ray diffractograms do not show the main reflex typical for anatase at 25.32 theta.

3. Further Processing

Fibers from the masses of example 1.1 were kept at 200° C. in air in a drying chamber. After a heating period of 2 h, several hours were required for 50 mg of fibers to dissolve in 10 mg of water at room temperature. After 24 h at 200° C., they were insoluble in water.

1. Polytitanic acid esters having linear cross-linking structures, obtainable by reacting a titanium compound that is soluble in an alcohol, in water or in a mixture of water and alcohol and that has the composition (I)
Ti(R)_{M}X_{N} (I),
wherein the groups R independently mean alkoxy, carboxy or OH, X means halogen, m=0 to 4, n=0 to 4 and m+n=4,
with a compound (II) that contains the following structural unit:
[O═CR⁴—(CR⁵R³)ₚ—CR¹R²—O]⁻,
wherein p is 0 or 1 and wherein
(a) R^{l} and R² are independently selected from among hydrogen, alkyl and alkenyl, R³ and R⁵ are independently selected from among hydrogen and an unsubstituted or substituted alkyl or alkenyl, R⁴ is OH or O(M^{x+})_{1/x}, wherein M is a metal or ammonium and x is 1 or 2,
or
(b) R^{l} and R² together mean ═O and R³, R⁵ and R⁴ are independently selected from among hydrogen, alkyl and alkenyl,
wherein the titanium compound (I) is reacted with compound (II) at a molar ratio of 1 to 0.5-1.9, with respect to an unbridged titanium compound, that is a (monomeric) titanium compound containing a single titanium atom, with the stipulation that, if compound (I) has no more than 2 hydroxy groups and if the reaction occurs in a water-free medium, the product formed by the reaction is then brought in contact with water in such a way that a hydrolytic condensation reaction of the produced titanium compound(s) occurs in all cases.

2. Polytitanic acid esters according to claim 1, wherein R in the compound (I) means alkoxy and/or X means chlorine.

3. Polytitanic acid esters according to claim 2, wherein R in the compound (I) means ethoxy and/or m=4.

4. Polytitanic acid esters according to claim 1, wherein at least one of the groups R³ and R⁵ is an alkyl substituted with a hydroxyl group.

5. Polytitanic acid esters according to claim 1, wherein the compound (II) is selected from among hydroxy carboxylic acids.

6. Polytitanic acid esters according to claim 5, wherein an ammonium compound, ammonia or an alkali or alkali earth salt is added to the mass during or after the hydrolytic condensation reaction.

7. Polytitanic acid esters according to claim 1, wherein the compound (II) is selected from among salts of hydroxy carboxylic acids.

8. Polytitanic acid esters according to claim 1, wherein the compound (II) is selected from among keto- and aldo-carboxylic acids.

9. Polytitanic acid esters according to claim 1, wherein the compound with the formula (II) is selected from among glycolic acid, lactic acid, 2-hydroxybutyric acid, 2-hydroxyisobutyric acid, 2,2-bis(hydroxymethyl)propionic acid, 2-oxopropanoic acid, glyoxylic acid and mixtures of these acids.

10. Polytitanic acid esters according to claim 1, obtainable with the further addition of acetic acid and/or propionic acid.

11. A spinnable mass, comprising a polytitanic acid ester according to claim 1 in a solvent, selected from among water, alcohols and ketones as well as mixtures of the aforementioned solvents.

12. A spinnable mass according to claim 11, wherein the solvent is selected from among water, methanol, ethanol, propanol, butanol as well as mixtures of the mentioned solvents.

13. A method for the manufacture of fibers containing titanium, characterized in that a viscous mass is provided that comprises a polytitanic acid ester as defined in claim 1, having a solid matter content of 20-50 percent by weight, preferably a solid matter content of 30-35 percent by weight, with respect to the theoretical titanium dioxide content in a solvent, said mass being extruded through spinning nozzles and the threads created in the process being subject to drying.

14. A method for the manufacture of fibers containing titanium which are poorly soluble or insoluble in water, characterized in that a viscous mass is provided that comprises a polytitanic acid ester as defined in claim 1, having a solid matter content of 20-50 percent by weight, preferably with a solid matter content of 30-35 percent by weight, in a solvent, said mass being extruded through spinning nozzles and the threads created in the process subsequently being heated for at least 5 hours at a temperature of at least 100° C., preferably at least 150° C.

15. A method according to claim 13, characterized in that the viscous mass was manufactured exclusively with the use of the titanium compound of compound (I) and the mentioned compound (II) as well as optionally with a monocarboxylic acid and otherwise only contains one or a plurality of suitable solvents.
